(19) United States
(12) Patent Application Publication
(10) Pub. No.: US 2024/0277415 A1
(43) Pub. Date: Aug. 22, 2024

(54) SYSTEM AND METHOD FOR MOVING A GUIDE SYSTEM

(71) Applicant: Mazor Robotics Ltd., Caesarea (IL)

(72) Inventor: Ido ZUCKER, Tel Aviv (IL)

(21) Appl. No.: 18/171,792

(22) Filed: Feb. 21, 2023

(51) Int. Cl. A61B 34/20; A61B 34/10

(52) U.S. Cl. CPC A61B 34/20; A61B 34/10; A61B 2034/105; A61B 2034/2072

(57) ABSTRACT

Disclosed is a system for assisting in guiding and performing a procedure on a subject. The subject may be any appropriate subject such as inanimate object and/or an animate object. The guide and system may include various manipulable or movable members, such as robotic systems, and may be registered to selected coordinate systems to assist in movement of the robotic systems.

### FIELD

The subject disclosure is related generally to a tracking and navigation system, and particularly to tracking a guide member and generating an a model.

### BACKGROUND

This section provides background information related to the present disclosure which is not necessarily prior art.

An instrument can be navigated relative to a subject for performing various procedures. For example, the subject can include a patient on which a surgical procedure is being performed. During a surgical procedure, an instrument can be tracked in an object or subject space. In various embodiments, the subject space can be a patient space defined by a patient. The location of the instrument that is tracked can be displayed on a display device relative to an image of the patient.

The position of the patient can be determined with a tracking system. Generally, a patient is registered to the image, via tracking an instrument relative to the patient to generate a translation map between the subject or object space (e.g., patient space) and the image space. This often requires time during a surgical procedure for a user, such as a surgeon, to identify one or more points in the subject space and correlating, often identical points, in the image space.

After registration, the position of the instrument can be appropriately displayed on the display device while tracking the instrument. The position of the instrument relative to the subject can be displayed as a graphical representation, sometimes referred to as an icon on the display device.

### SUMMARY

This section provides a general summary of the disclosure, and is not a comprehensive disclosure of its full scope or all of its features.

According to various embodiments, a selected volume that may include a subject, a fiducial object and/or other portions can be imaged with an imaging system. The imaging system may collect image data. The image data may be used to generate a model. The model may have selected clarity and/or resolution including within selected portions of the model.

A robotic system may include an appropriate robotic system, such as a Mazor X™ Robotic Guidance System, sold by Mazor Robotics Ltd. having a place of business in Israel and/or Medtronic, Inc. having a place of business in Minnesota, USA. The robotic system may include the fiducial object that is imaged with the subject and may include one or more objects, such as an array of discrete objects. The discrete objects may include one or more shapes, such as spheres, cubes, one or more rods that can all be in one or intersect one plane, etc. The fiducial object can be modeled in three-dimensional (3D) space as a 3D model. Fiducial features can be extracted from the 3D model. The fiducial features can be compared to or coordinated with image fiducial features that are the imaged fiducial object or some portion thereof (e.g., an image fiducial feature can be a point relating to a center of a sphere or a circle or point relating to an intersection of a rod with a plane).

In various embodiments, the different systems used relative to the subject may include different coordinate systems (e.g., locating systems). For example, a robotic system may be moved relative to a subject that includes a robotic coordinate system. The robot system may include a robot portion (e.g., a robotic arm, robotic joint, robot end effector) that may be fixed, including removably fixed, at a position relative to the subject. Thus, movement of a portion of the robot system relative to a base of the robot system (i.e. the fixed portion of the robot system) may be known due to various features of the robot. For example, encoders (e.g., optical encoders, potentiometer encoders, or the like) may be used to determine movement or amount of movement of various joints (e.g., pivots, joints) of a robot. A position of an end effector (e.g., a terminal end) of the robot may be known relative to the base of the robot. Given a known position of the subject relative to the base and the immovable relative position of the base and the subject, the position of the end effector relative to the subject may be known during movement of a robot and/or during a stationary period of the end effector. Thus, the robot may define a coordinate system relative to the subject.

Various other portions may also be tracked relative to the subject. For example, a tracking system may be incorporated into a navigation system that includes one or more instruments that may be tracked relative to the subject. The navigation system may include one or more tracking systems that track various portions, such as tracking devices, associated with instruments. The tracking system may include a localizer that is configured to determine the position of the tracking device in a navigation system coordinate system. Determination of the navigation system coordinate system may include those described at various references including U.S. Pat. Nos. 8,737,708; 9,737,235; 8,503,745; and 8,175,681; all incorporated herein by reference. In particular, a localizer may be able to track an object within a volume relative to the subject. The navigation volume, in which a device, may be tracked may include or be referred to as the navigation coordinate system or navigation space. A determination or correlation between the two coordinate systems may allow for or also be referred to as a registration between two coordinate systems. In addition, one or more portions of a robotic system may be tracked with the navigation system. Thus, the navigation system may track the robot and the subject in the same coordinate system with selected tracking devices.

In various embodiments the first coordinate system, which may be a robotic coordinate system, may be registered to a second coordinate system, which may be a navigation coordinate system. Accordingly, coordinates in one coordinate system may then be transformed to a different or second coordinate system due to a registration also referred to as a translation map in various embodiments. Registration may allow for the use of two coordinate systems and/or the switching between two coordinate systems. For example, during a procedure a first coordinate system may be used for a first portion or a selected portion of a procedure and a second coordinate system may be used during a second portion of a procedure. Further, two coordinate systems may be used to perform or track a single portion of a procedure, such as for verification and/or collection of additional information.

Furthermore, image data and/or images may be acquired of selected portions of a subject. Image data may be used to generate or reconstruct a model of the subject, such as a 3D (i.e., volumetric) model of the subject. The model and/or other images may be displayed for viewing by a user, such as a surgeon. The superimposed on a portion of the model or image may be a graphical representation of a tracked portion or member, such as an instrument. According to various embodiments, the graphical representation may be superimposed on the model or image at an appropriate position due to registration of an image space (also referred to as an image coordinate system) to a subject space. A method to register a subject space defined by a subject to an image space may include those disclosed in U.S. Pat. Nos. 8,737,708; 9,737,235; 8,503,745; and 8,175,681; all incorporated herein by reference. Discussed herein, the image displayed may be displayed on a display device. The image may be a direct image (e.g., visible image, 2D x-ray projection), a model reconstructed based on selected data (e.g., a plurality of 2D projections, a 3D scan (e.g., computer tomography), magnetic resonance image data), or other appropriate image. Thus, the image as referred to therein that is displayed may be reconstructed or a raw image.

During a selected procedure, the first coordinate system may be registered to the subject space or subject coordinate system due to a selected procedure, such as imaging of the subject. In various embodiments, the first coordinate system may be registered to the subject by imaging the subject with a fiducial portion that is fixed relative to the first member or system, such as the robotic system. The known position of the fiducial relative to the robotic system may be used to register the subject space relative to the robotic system due to the image of the subject including the fiducial portion. Thus, the position of the robotic system or a portion thereof, such as the end effector, may be known or determined relative to the subject. Due to registration of a second coordinate system to the robotic coordinate system may allow for tracking of additional elements not fixed to the robot relative to a position determined or tracked by the robot.

The tracking of an instrument during a procedure, such as a surgical or operative procedure, allows for navigation of a procedure. When image data is used to define an image space it can be correlated or registered to a physical space defined by a subject, such as a patient. According to various embodiments, therefore, the patient defines a patient space in which an instrument can be tracked and navigated. The image space defined by the image data can be registered to the patient space defined by the patient. The registration can occur with the use of fiducials that can be identified in the image data and in the patient space.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is diagrammatic view illustrating an overview of a robotic system and a navigation system, according to various embodiments;

FIG. 2 is a detailed environmental view of a robotic system and a tracking system with the robotic system in a first configuration, according to various embodiments;

FIG. 3 is a schematic view of an imaging system to acquired image data of a subject, according to various embodiments;

FIG. 4 is a schematic view of an imaging system to acquired image data of a subject, according to various embodiments;

FIG. 5 is a detailed environmental view of the robotic system and the tracking system with the robotic system in a second configuration, according to various embodiments; and

FIG. 6 is a flow chart of a method or process to determine a Go Zone for movement of the robotic system, according to various embodiments.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein. In various embodiments, it is understood, that the systems and methods may be incorporated into and/or used on non-animate objects. The systems may be used to, for example, to register coordinate systems between two systems for use on manufacturing systems, maintenance systems, and the like. For example, automotive assembly may use one or more robotic systems including individual coordinate systems that may be registered together for coordinated or consorted actions. Accordingly, the exemplary illustration of a surgical procedure herein is not intended to limit the scope of the appended claims.

Discussed herein, according various embodiments, are processes and systems for allowing registration between various coordinate systems. In various embodiments, a first coordinate system that may be a robotic coordinate system may be registered to a second coordinate system that may be an image coordinate system or space. A third coordinate space, such as a navigation space or coordinate system, may then be registered to the robotic or first coordinate system and, therefore, be registered to the image coordinate system without being separately or independently registered to the image space. Similarly, the navigation space or coordinate system may be registered to the image coordinate system or space directly or independently. The robotic or first coordinate system may then be registered to the navigation space and, therefore, be registered to the image coordinate system or space without being separately or independently registered to the image space.

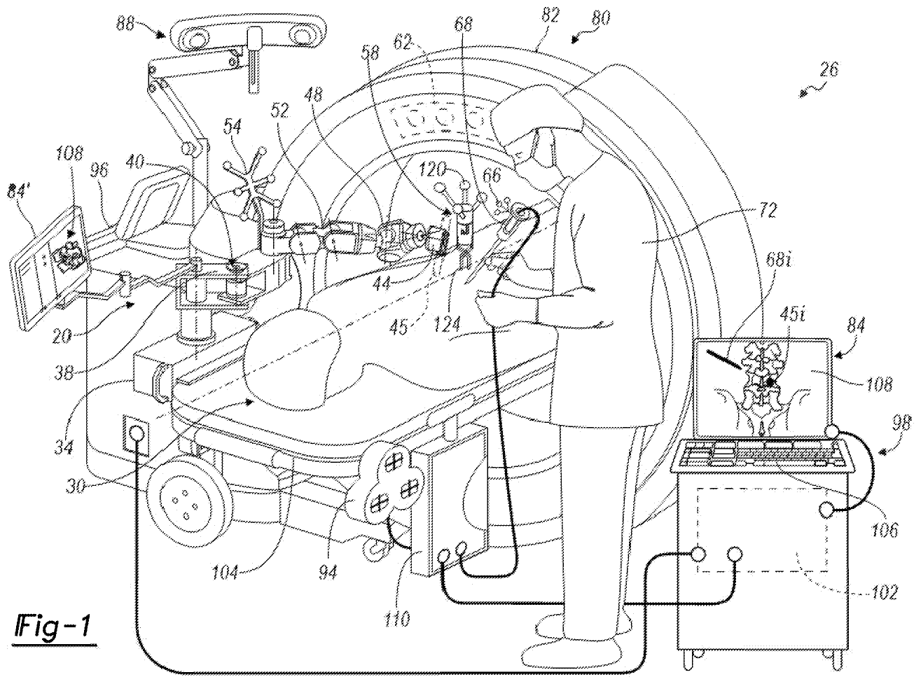

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding selected instrument, such as drills, screws, etc. relative to a subject 30. The robotic system 20 may include a mount 34 that fixes a portion, such as a robotic base 38, relative to the subject 30. The robotic system 20 may include one or more arms 40 that are moveable or pivotable relative to the subject 30, such as including an end effector 44. The end effector may be any appropriate portion, such as a tube, guide, or passage member. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20.

The navigation system 26 can be used to track the location of one or more tracking devices, tracking devices may include a robot tracking device 54, a subject tracking device 58, an imaging system tracking device 62, and/or a tool tracking device 66. A tool 68 may be any appropriate tool such as a drill, forceps, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An imaging device 80 may be used to acquire pre-, intra-, or post-operative or real-time image data of a subject, such as the subject 30. The image data may be used to reconstruct or generate an image of the subject and/or various portions of the subject or space relative to the subject 30. Further, the image data may be used to generate models of more than one resolution, as discussed herein. The models may be used for various purposes, such as determining a region for movement of the end effector 44 and/or other portions of the robotic arm 40.

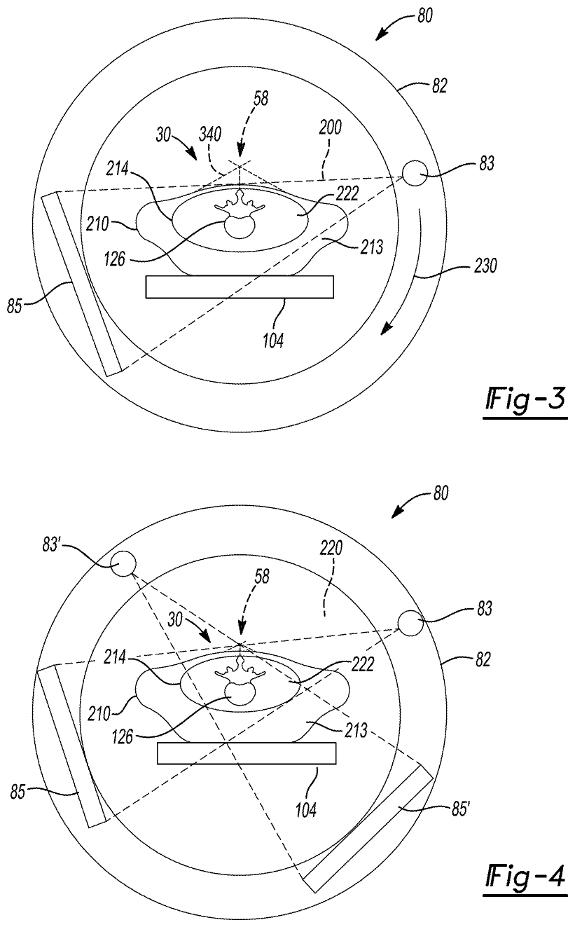

It will be understood, however, that any appropriate subject can be imaged and any appropriate procedure may be performed relative to the subject. In the example shown, the imaging device 80 comprises an O-arm® imaging device sold by Medtronic Navigation, Inc. having a place of business in Louisville, Colorado, USA. The imaging device 80 may have a generally annular gantry housing 82 in which an image capturing portion is moveably placed. The image capturing portion may include an x-ray source or emission portion 83 (FIGS. 3 and 4) and an x-ray receiving or image receiving portion 85 (FIGS. 3 and 4) located generally or as practically possible 180 degrees from each other and mounted on a rotor relative to a track or rail. The image capturing portion can be operable to rotate 360 degrees during image acquisition. The image capturing portion may rotate around a central point or axis, allowing image data of the subject 80 to be acquired from multiple directions or in multiple planes. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. In one example, the imaging device 80 can utilize flat plate technology having a 1,720 by 1,024 pixel image data capture area.

The position of the imaging device 80, and/or portions therein such as the image capturing portion, can be precisely known relative to any other portion of the imaging device 80. The imaging device 80, according to various embodiments, can know and recall precise coordinates relative to a fixed or selected coordinate system. This can allow the imaging system 80 to know its position relative to the patient 30 or other points in space. In addition, as discussed herein, the precise knowledge of the position of the image capturing portion can be used in conjunction with a tracking system to determine the position of the image capturing portion and the image data relative to the tracked subject, such as the patient 30.

The imaging device 80 can also be tracked with a tracking device 62. The image data defining an image space acquired of the patient 30 can, according to various embodiments, be inherently or automatically registered relative to an object space. The object space can be the space defined by a patient 30 in the navigation system 26. The automatic registration can be achieved by including the tracking device 62 on the imaging device 80 and/or the determinable precise pose (i.e., including at least three degree of freedom location information (e.g., x, y, and z coordinates) and/or three degree of freedom orientation information) of the image capturing portion. According to various embodiments, as discussed herein, imageable portions, virtual fiducial points and other features can also be used to allow for registration, automatic or otherwise. It will be understood, however, that image data can be acquired of any subject which will define subject space. Patient space is an exemplary subject space. Registration allows for a translation map to be determined between patient space and image space.

The patient 80 can also be tracked as the patient moves with a patient tracking device, DRF, or tracker 58. Alternatively, or in addition thereto, the patient 30 may be fixed within navigation space defined by the navigation system 26 to allow for registration. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84. Various tracking systems, such as one including an optical localizer 88 or an electromagnetic (EM) localizer 92 can be used to track the instrument 68.

More than one tracking system can be used to track the instrument 68 in the navigation system 26. According to various embodiments, these can include an electromagnetic (EM) tracking system having the EM localizer 94 and/or an optical tracking system having the optical localizer 88. Either or both of the tracking systems can be used to track selected tracking devices, as discussed herein. It will be understood, unless discussed otherwise, that a tracking device can be a portion trackable with a selected tracking system. A tracking device need not refer to the entire member or structure to which the tracking device is affixed or associated.

It is further appreciated that the imaging device 80 may be an imaging device other than the O-arm® imaging device and may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

In various embodiments, an imaging device controller 96 may control the imaging device 80 and can receive the image data generated at the image capturing portion and store the images for later use. The controller 96 can also control the rotation of the image capturing portion of the imaging device 80. It will be understood that the controller 96 need not be integral with the gantry housing 82, but may be separate therefrom. For example, the controller may be a portions of the navigation system 26 that may include a processing and/or control system 98 including a processing unit or processing portion 102. The controller 96, however, may be integral with the gantry 82 and may include a second and separate processor, such as that in a portable computer.

The patient 30 can be fixed onto an operating table 104. According to one example, the table 104 can be an Axis Jackson® operating table sold by OSI, a subsidiary of Mizuho Ikakogyo Co., Ltd., having a place of business in Tokyo, Japan or Mizuho Orthopedic Systems, Inc. having a place of business in California, USA. Patient positioning devices can be used with the table, and include a Mayfield® clamp or those set forth in commonly assigned U.S. patent application Ser. No. 10/405,068 entitled “An Integrated Electromagnetic Navigation And Patient Positioning Device”, filed Apr. 1, 2003 and published as U.S. Pat. App. Pub. No. 2004/0199072, which is hereby incorporated by reference.

The position of the patient 30 relative to the imaging device 80 can be determined by the navigation system 26. The tracking device 62 can be used to track and locate at least a portion of the imaging device 80, for example the gantry or housing 82. The patient 30 can be tracked with the dynamic reference frame 58, as discussed further herein. Accordingly, the position of the patient 30 relative to the imaging device 80 can be determined. Further, the location of the imaging portion can be determined relative to the housing 82 due to its precise position on the rail within the housing 82, substantially inflexible rotor, etc. The imaging device 80 can include an accuracy of within 10 microns, for example, if the imaging device 80 is an O-Arm® imaging device sold by Medtronic Navigation, Inc. having a place of business in Louisville, Colorado. Precise positioning of the imaging portion is further described in U.S. Pat. Nos. 7,188,998; 7,108,421; 7,106,825; 7,001,045; and 6,940,941; all of which are incorporated herein by reference,

According to various embodiments, the imaging device 80 can generate and/or emit x-rays from the x-ray source that propagate through the patient 30 and are received by the x-ray imaging receiving portion. The image capturing portion generates image data representing the intensities of the received x-rays. Typically, the image capturing portion can include an image intensifier that first converts the x-rays to visible light and a camera (e.g., a charge coupled device) that converts the visible light into digital image data. The image capturing portion may also be a digital device that converts x-rays directly to digital image data for generating or reconstructing images, thus potentially avoiding distortion introduced by first converting to visible light.

Two dimensional and/or three dimensional fluoroscopic image data that may be taken by the imaging device 80 can be captured and stored in the imaging device controller 96. Multiple image data taken by the imaging device 80 may also be captured and assembled to provide a larger view or image of a whole region of a patient 30, as opposed to being directed to only a portion of a region of the patient 30. For example, multiple image data of the patient's 30 spine may be appended together to provide a full view or complete set of image data of the spine.

The image data can then be forwarded from the image device controller 96 to the navigation computer and/or processor system 102 that can be a part of a controller or work station 98 having the display 84 and a user interface 106. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. Pat. No. 7,751,865, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Louisville, Colorado. Other tracking systems include acoustic, radiation, radar, etc. tracking systems. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Various portions of the navigation system 26, such as the instrument 68, and others as will be described in detail below, can be equipped with at least one, and generally multiple, of the tracking devices 66. The instrument can also include more than one type or modality of tracking device 66, such as an EM tracking device and/or an optical tracking device. The instrument 68 can include a graspable or manipulable portion at a proximal end and the tracking devices may be fixed near the manipulable portion of the instrument 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track the instrument 68 relative to the patient 30. The instrument 68 can be tracked with the tracking system, as discussed above. Image data of the patient 30, or an appropriate subject, can be used to assist the user 72 in guiding the instrument 68. The image data, however, is registered to the patient 30. The image data defines an image space that is registered to the patient space defined by the patient 30. The registration can be performed as discussed herein, automatically, manually, or combinations thereof. The tracking system may also be used to track the robotic system 20, or at least a portion thereof such as the movable portions including the arm 40 and/or the end effector 44. In various embodiments, a registration or translation map of the robotic coordinate system and the coordinate system defined by the subject may be made to determine a volume in which the robotic system may move. This may be done, at least in part, due to the registration of the subject and image space.

Generally, registration allows a translation map to be generated of the physical location of the instrument 68 relative to the image space of the image data. The translation map allows the tracked position of the instrument 68 to be displayed on the display device 84 relative to the image data 108. A graphical representation 68i, also referred to as an icon, can be used to illustrate the location of the instrument 68 relative to the image data 108.

This may also allow for registration or a translation map to be determined between various other coordinate system that relate to other portions, such as the robotic coordinate system of the robotic system 20. For example, once the navigation coordinate system is determined in physical space, and a registration is made to the image space, any portion that is tracked in the navigation space may be tracked relative to the subject space as may any other portion that has a translation map to allow for registration between a separate coordinate space and the image coordinate space. As discussed above the registration to the navigation space may be maintained with respect to the subject 30 by maintaining a tracking device on the subject 30. In the alternative and/or additionally, the subject 30 may be fixed in space relative to the navigation coordinate system.

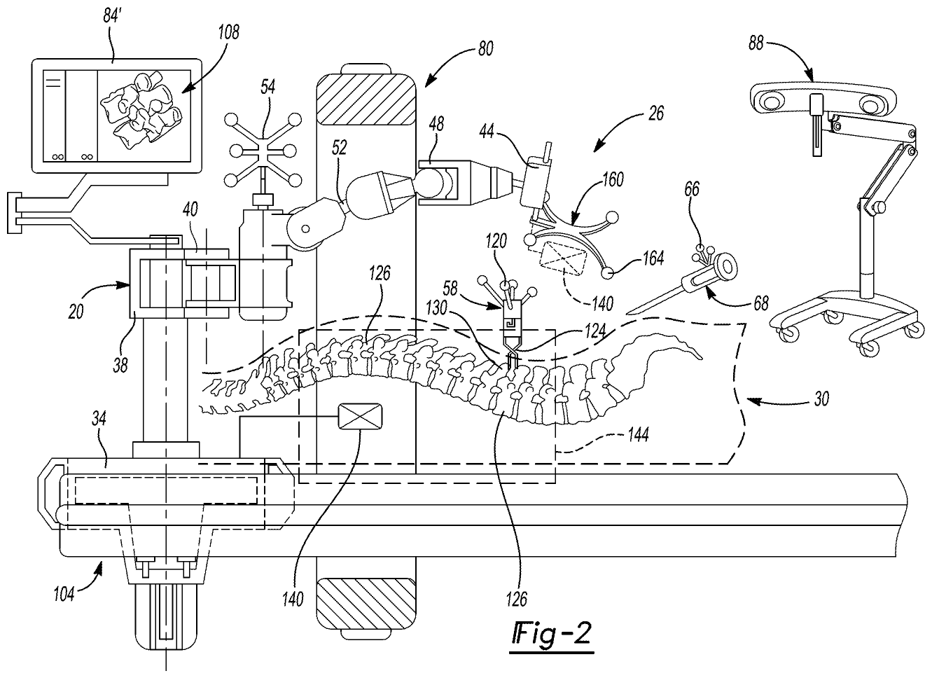

According to various embodiments, a subject registration system or method can use the tracking device 58 may include that as disclosed in U.S. Pat. No. 11,135,025, incorporated herein by reference. Briefly, with reference to FIG. 1 and FIG. 2. The tracking device 58 may include portions or members 120 that may be trackable, but may also act as or be operable as a fiducial assembly. The fiducial assembly 120 can include a clamp or other fixation portion 124 and the imageable fiducial body 120. It is understood, however, that the members 120 may be separate from the tracking device 58. The fixation portion 124 can be provided to fix any appropriate portion, such as a portion of the anatomy. As illustrated in FIGS. 1 and 2, the fiducial assembly 120 can be interconnected with a portion of a spine 126 such as a spinous process 130.

The fixation portion 124 can be interconnected with the spinous process 130 in any appropriate manner. For example, a pin or a screw can be driven into the spinous process 130. Alternatively, or in addition thereto, a clamp portion 124 can be provided to interconnect the spinous process 130. The fiducial portions 120 may be imaged with the imaging device 80. It is understood, however, that various portions of the subject (such as a spinous process) may also be used as a fiducial portion.

In various embodiments, when the fiducial portions 120 are imaged with the imaging device 80, image data is generated that includes or identifies the fiducial portions 120. The fiducial portions 120 can be identified in image data automatically (e.g., with a processor executing a program), manually (e.g., by selection an identification by the user 72), or combinations thereof (e.g., by selection an identification by the user 72 of a seed point and segmentation by a processor executing a program). Methods of automatic imageable portion identification include those disclosed in U.S. Pat. No. 8,150,494 issued on Apr. 3, 2012, incorporated herein by reference. Manual identification can include selecting an element (e.g., pixel) or region in the image data wherein the imageable portion has been imaged. Regardless, the fiducial portions 120 identified in the image data can be used as fiducial points or positions that can be used to register the image data or the image space of the image data with patient space.

In various embodiments, to register an image space or coordinate system to another space or coordinate system, such as a navigation space, the fiducial portions 120 that are identified in the image 108 may then be identified in the subject space defined by the subject 30, in an appropriate manner. For example, the user 72 may move the instrument 68 relative to the subject 30 to touch the fiducial portions 120, if the fiducial portions are attached to the subject 30 in the same position during the acquisition of the image data to generate the image 108. It is understood that the fiducial portions 120, as discussed above in various embodiments, may be attached to the subject 30 and/or may include anatomical portions of the subject 30. Additionally, a tracking device may be incorporated into the fiducial portions 120 and they may be maintained with the subject 30 after the image is acquired. In this case, the registration or the identification of the fiducial portions 120 in a subject space may be made. Nevertheless, according to various embodiments, the user 72 may move the instrument 68 to touch the fiducial portions 120. The tracking system, such as with the optical localizer 88, may track the position of the instrument 68 due to the tracking device 66 attached thereto. This allows the user 72 to identify in the navigation space the locations of the fiducial portions 120 that are identified in the image 108. After identifying the positions of the fiducial portions 120 in the navigation space, which may include a subject space, the translation map may be made between the subject space defined by the subject 30 in a navigation space and the image space defined by the image 108. Accordingly, identical or known locations allow for registration as discussed further herein.

During registration, a translation map is determined between the image data coordinate system of the image data (which may be used to generate or reconstruct the image 108) and the patient space defined by the patient 30. Once the registration occurs, the instrument 68 can be tracked with the tracking system that is registered to the image data to allow an identification and illustration of a position of the tracked instrument 68 as an icon superimposed on the image data. Registration of the image 108 (or any selected image data) to the subject 30 may occur at any appropriate time.

After the registration of the image space to the patient space, the instrument 68 can be tracked relative to the image 108. As illustrated in FIG. 1, the icon 68i representing a position (which may include a six-degree of freedom position (including 3D location and orientation)) of the instrument 68 in the navigation space can be displayed relative to the image 108 on the display 84. Due to the registration of the image space to the patient space, the position of the icon 68i relative to the image 108 can substantially identify or mimic the location of the instrument 68 relative to the patient 30 in the patient space. As discussed above, this can allow a navigated procedure to occur.

The robotic system 20 having the robotic system coordinate system may be registered to the navigation space coordinate system, as discussed herein, due to the reference tracking device 54 (e.g., if positioned and/or fixed to a known position on or relative to the robotic system 20) and/or due to the tracking of the snapshot tracking device 160. The snapshot tracking device 160 may include one or more trackable portions 164 that may be tracked with the localizer 88 or any appropriate localizer (e.g. optical, EM, radar). It is understood, however, that any appropriate tracking system may be used to track the snapshot tracking device 160. The reference tracking device 54 may be fixed relative to a selected portion of the robotic system 20 to be tracked during a procedure and/or the snapshot tracking device 160 may be connected to a portion of the robotic system 20 during a registration procedure. After the registration, the pose of selected portions of the robotic system 20 may be determined with a tracking device (e.g., the tracking device 54) and/or with various sensors incorporated with the robotic system, such as position sensors incorporated with motors or movement system of the robotic system 20.

A fixed reference tracking device may also be positioned within the navigation space. The fixed navigation tracker may include the patient tracker 58 which may be connected to the patient 30 and/or the robot tracker 54 that may be fixed to the robotic system 20, such as the base 34. The reference tracker, therefore, may be any appropriate tracker that is positioned alternatively to and/or in addition to the snapshot tracker 160 that is within the navigation coordinate space during the registration period. For the discussion herein the robot tracker 54 will be referred to however, the patient tracker 58 may also be used as the reference tracker. Further, reference tracker may be positioned within the coordinate system at any position in addition or alternatively to and relative to the snapshot tracker 160 as long as the snapshot tracker 160 may be tracked relative to the reference tracker.

In various embodiments, the snapshot tracker 160 may be positioned at a known position relative to the end effector 44. For example, the snapshot tracker 160, which includes the trackable portions 164, extends from a rod or connection member 168. The localizer 88 may then view or determine a position of the snapshot tracking device 160 relative to the reference tracking device 54 and or the reference tracking device 58. As the localizer 88 defines or may be used to define the navigation space, determining or tracking a position of the snapshot tracker 160 relative to the reference frame 54 may be used to determine a relationship between a position within the navigation space and the robotic space of the end effector 44. The snapshot tracker 160 may be used during registration and/or may be positioned relative to the end effector during any movement of the robotic system 20. In various embodiments, the snapshot tracker 160 may be provided at or near the end effector to track the end effector in addition to any instrument placed to be used with the end effector 44.

With reference to FIG. 1 and FIG. 2 and additional reference to FIG. 3 and FIG. 4, the imaging system 20 may be used to capture image data of the subject 30. The image data captured of the subject 30 may be captured due to the emission of x-rays from the source 83 and detection at the detector 85. It is understood by one skilled in the art, the x-rays may be attenuated and/or blocked by one or more portions of the subject 30 and other x-rays may pass through portions of the subject 30. The detected x-rays or attenuation of the x-rays at the detector 85 may generate that is used to generate or reconstruct images that may be displayed, such as the image 108 of the display device 84. It is understood, therefore, that image data may be acquired of the subject 30 and images and/or models may be generated based upon the image data. The models may be generated to include various information and/or features that may include only direct image data of the subject and/or other data.

According to various embodiments, as illustrated in FIG. 3, the imaging system 80 may include the source 83 that generates a beam 200 of x-rays, such as within a cone or triangle, the x-rays may be generated at the source 83 and detected at the detector 85. The subject 30 may be positioned within the beam 200 of x-rays and attenuate and/or block them before they contact the detector 85. The subject 30 may include an outer or external geometry 210. The outer geometry 210 may be an outer boundary of the subject 30. The subject 30 may also include various internal geometry or portions, such as the spine 126. The subject 30 may also be separated into various portions including a region between the outer extent 210 and the spine 126, including intermediate boundaries 214. Further regions may be defined between the intermediate boundary 214 and the spine 126. A first area 213 and a second area 222 may have image data captured at the detector 85 in a substantially similar manner as image data is detected regarding any other portion, such as the spine 126. During a reconstruction of the image data captured at the detector 85, however, the various regions may be reconstructed in an image and/or model at different resolutions. The various resolutions may assist in speed of generation and/or information used for the reconstruction. Further, it may be selected to only display various portions of a reconstruction while allowing for a model or reconstruction of various portions of the subject 30, such as the outer extent 210, to be made and used for other purposes that do not include display thereof.

As illustrated in FIG. 3, the cone 200 may capture an entire volume of the subject 30 at a single time. The imaging system 80 may be any appropriate imaging system, such as the O-Arm® imaging system, as discussed above. Accordingly, source 80 and detector 83, 85 may rotate within the gantry 82, such as generally the direction of arrow 230. Images or image data may be captured of the subject 30 at various angles relative to the subject 30. In various embodiments, the subject 30 may be substantially fixed or unmoving during the capture of image data thereof such that a plurality of image projections may be acquired of the subject 30. This may assist in a three-dimensional reconstruction of the subject 30 at a selected time. In various embodiments, for example, the subject 30 may be placed on and/or fixed to the support 104.

With reference to FIG. 4, the imaging system 80 may acquire image data of the subject 30 when the subject 30 is not in an iso-center of the imaging system and/or during an eccentric image capture. As illustrated in FIG. 4, The beam 200 may be emitted from the source 83 and detected at the detector 85. As illustrated in FIG. 4, however, the entire subject 30 may not be within the beam 200 at any single detection position. Nevertheless, as discussed above, the detector 85 and the source 83 may move within the gantry to various positions to allow for capture of perspectives of the subject 30. For example, is illustrated in FIG. 4, image data may be a captured with at least two positions of the source 83 and 83′ and two positions of the detector 85, 85′. The plurality of projections of the subject 30 may be used to generate or reconstruct selected images and/or models of the subject 30. Therefore, regardless of the position of the subject 30 within the imaging system 80, selected image data may be acquired and used to reconstruct images or models of the subject 30. Further, as illustrated in FIG. 4, the various portions of the subject 30 may be imaged or reconstructed, including the outer boundary 210, the intermediate boundary 214, and the spine 216. The first portion 213 and the second portion 222 may, however, as discussed above, also be reconstructed at the various resolutions for various purposes.

As discussed above, the imaging system 80 may acquire image data of the subject 30. The image data of the subject 30 may include image data of all portions of the subject 30, at least for a selected area or region of interest. For example, as illustrated in FIG. 2, the spine 126 may be image data of the subject 30. The image data acquired of the subject 30 may also include soft tissue image data of tissue adjacent to the vertebrae of the spine 126. The image data acquired with the imaging system 80 may be used to identify various portions of the anatomy of the subject 30 including the spine 126 and other portions. In various embodiments, reconstruction of the spine 126 may be made and displayed, or at least a portion thereof, as the image 108. The image data of portions relative to the spine 126 may also be captured in the image data with the imaging system 80. This image data may be used to generate or reconstruct a model of one or more portions of the subject 30 including the spine 126.

In various embodiments, the spine 126 may be reconstructed with a high degree of resolution, such as a maximum possible resolution. In various embodiments, the high resolution may be about 100 pixels per inch, 75 pixels per inch or any appropriate number. Therefore, images of the spine may be displayed with detail to allow for a procedure to occur relative to the spine or portions of the spine 126. As illustrated in FIG. 2, the image 108 may include a detailed view of at least a single vertebrae of the spine 126. Other portions of the subject 30, however, may be reconstructed with a lower resolution. A lower resolution may be about 10 pixels per inch, 20 pixels per inch or any appropriate amount. In various embodiments, a low or lower resolution may be a resolution that is about 10% to 50% of the higher resolution.

As discussed above, an outer extent or boundary 210 of the subject 30 may be reconstructed with a low resolution and an intermediate region relative to an intermediate portion 214 may be reconstructed with an intermediate resolution, such as 40% to about 70% of the high resolution. In various embodiments, however, the resolution of an outer extent of the subject 30 may be reconstructed with any resolution operable to assist in defining a No-Go zone for portions of the procedure, such as for moving or placing the robotic system 40. The highest resolution portion may be used to reconstruct various portions of the subject, such as the spine 126. Therefore, image data may be reconstructed at selected resolutions to achieve a speed of reconstruction, resolution or reconstruction, for various purposes, such as those discussed further herein.

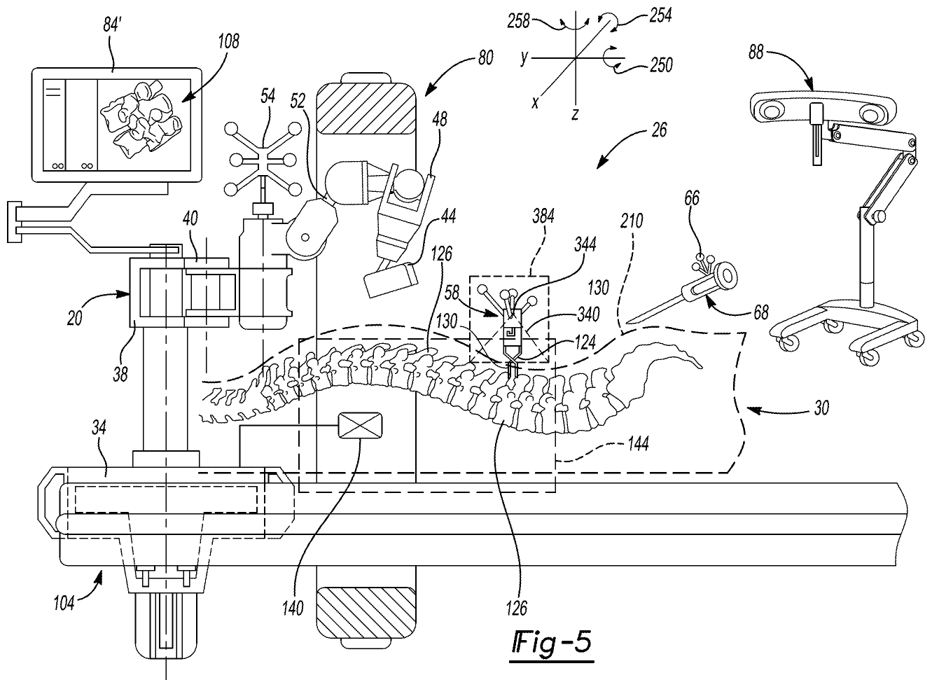

Turning reference to FIG. 5, the robotic system 20 includes the arm portion 40 including or having a moving portion. The end effector 44 may be the moving portion that moves relative to the subject 30. As illustrated in FIG. 2, the end effector 44 may be positioned in a first pose relative to the subject 30. The end effector 44 may be positioned relative to the patient tracking device 58 and the spine 126 at the first pose. During a procedure, such as after a first portion of a procedure, the end effector 44 may be selected to be moved to a second position or pose. Moving the end effector 44 to a second pose may allow for the end effector 44 to be positioned relative to a different or second portion of the subject 30 to assist in the procedure, especially at a second time. Therefore, the end effector 44 may be made to move or traverse relative to the subject 30.

While moving the end effector 44 relative to the subject 30, it is selected to have the end effector 44, or any portion of the robotic arm 40, not contact the subject 30. It may also be selected to have no portion of the robotic arm 40 contact any other portions or items in the navigation space. Therefore, the pose of the subject 30 may be determined and the volume of the subject 30 including an outer boundary of the subject that may include soft tissue of the subject 30. The soft tissue volume of the subject 30 may include a volume or region which is selected to have the robotic arm 40, including the end effector 44, not pass-through or contact. Therefore all portions of the subject, including the soft tissue thereof, may be determined are defined as a No-Go zone or region for movements of the robotic arm 40 including the end effector 44. As discussed herein, the No-Go Zone may be the volume in which the robotic arm 40 is selected to not move or be positioned.

The image data acquired of the subject 30 may include image data for all portions of the subject 30. The subject 30 may include the bone tissue, such as of the spine 126, and soft tissue relative to the spine 126. Therefore, the image data acquired with the imaging system 80 may be used to generate a model of the subject 30 in addition to the hard or bone tissue. The model may be used to define the No-Go region. Thus, when the robotic arm moves relative to the subject 30, the robotic arm may be controlled and instructed to not passed through various No-Go regions.

The robotic arm 40 may move relative to the subject 30 or anywhere in physical space in various degrees of freedom. In various embodiments, the robotic arm 40 may move in substantially 6 degrees of freedom. The 6 degrees of freedom of may include three axes of freedom X, Y, and Z axes, as illustrated in FIG. 5. In addition, freedom of movement of the robotic arm, including the end effector 44, may include orientation such as orientations around each of the three axes including a first rotational or annular orientation 250 relative to the X axis, a second orientation 254 relative to the Y axis, any third orientation 258 relative to the Z axis. Therefore, the robotic arm 44, including the end effector 44, may move relative to the subject 30 in any appropriate manner while not passing through a selection volume, which may also be referred to as a NO-Go Zone or region. The robotic arm may include any appropriate drive system to move, such as one or more electric motors, cable drive, belt drive, etc.

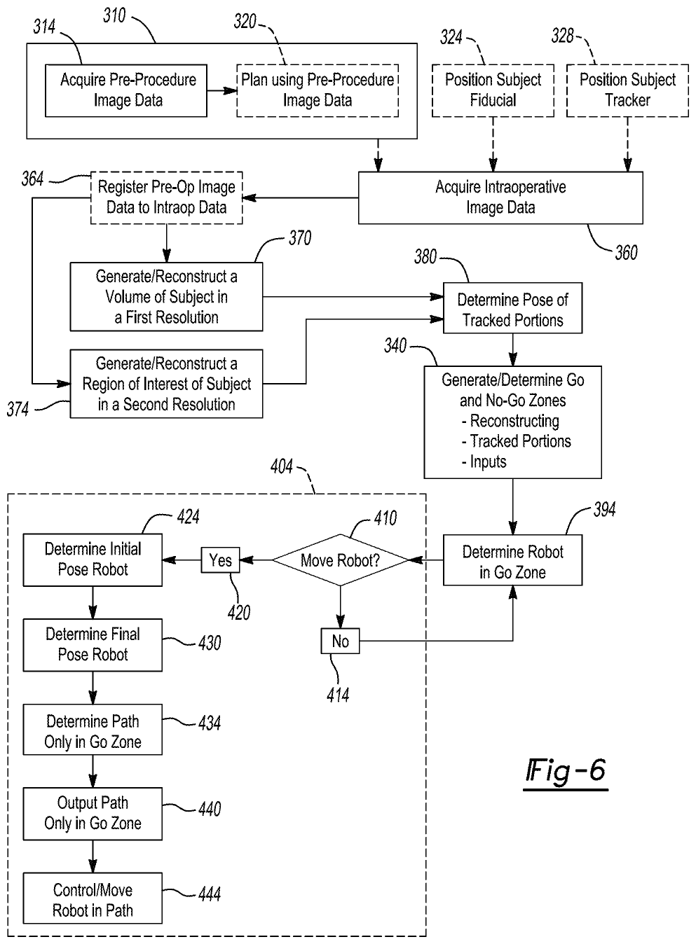

With reference to FIG. 6, a process or method 300 for acquiring data of the subject and determining a possible zone of movement of the robotic system 20 is disclosed. As discussed herein, the robotic system may move relative to the subject 30. Generally, however, they may be selected to have the robotic system 20 move only in a selected zone. The selected zone may be a volume defined in space that may also be referred to as a Go Zone or region. Generally, the Go Zone allows robotic system 20 be moved without contacting another object, such as the subject 30. Not included in the Go Zone may be a NO-GO Zone or region. Generally, the No-Go Zone may be defined any anything not in the Go Zone. As discussed herein, moving in and/or only moving in the Go Zone includes not moving in the No-Go Zone.

Image data or volume data relative to the subject 30 may be used to assist in defining the No-Go Zone. A No-Go Zone may include areas or volumes that would include create contact of the subject 30 by the robotic system 20. Further, as discussed above, various portions may be tracked with the navigation system. Therefore, the tracked portions may also be identified within the No-Go Zone to assist in ensuring that the robotic system does 20 not contact any portion during movement of the robotic arm 40. As discussed herein, any portion of the robotic system that may move may be a moveable portion, including the arm 40, the end effector 44, and/or other portion able to move relative to the base 34 and/or move relative to the subject 30.

The method 300 illustrated in FIG. 6 may include various inputs. In various embodiments, for example, an initial input or subroutine may include a subroutine 310 that includes acquiring pre-procedure or pre-operative image data in block 314. In various embodiments, a plan may be made with the pre-procedure image data in block 320. The pre-procedure image data acquired in block 314 may include magnetic residence image (MRI) data, computed tomography (CT) image data, cone beam image data, fluoroscopy image data, or other types of appropriate image data. The image data may be acquired of the subject 30 to assist in performing a procedure on the subject 30. The image data or other appropriate data in addition to and/or alternatively to image data may be acquired. The image data may be used to plan a procedure. For example, planning the procedure may include determining where an implant is to be positioned, a size or geometry of an implant, a location of an incision, or other appropriate procedure processes. Nevertheless, the subroutine 310 may include at least acquiring pre-procedure or preoperative image data that may be used during a procedure, as discussed further herein.

Other inputs and/or procedures may occur relative to the method 300. For example, a subject to may be positioned with a fiducial in block 324. Placing a fiducial in a subject 30 may include positioning and/or or fixing to the subject 30 an imageable portion. The fiducial may be imaged in the pre-procedure image data and/or intraoperative image data. The fiducial may be fixed to various portions, such as the robotic system 20, to assist in determining a pose of the robotic system 20, including the robotic arm 40, relative to the subject 30. In various embodiments, for example in the MazorX® robotic system and procedure, the fiducial may be imaged intraoperatively to determine a source or origin for movement of the robotic system 20 relative to the subject 30 and/or other portions. The fiducial may be used to define the origin of the base 34 in the image data based on the image fiducial. The fiducial may also be positioned on the subject such that it may be imaged during an image to allow for registration of the subject to the image data after acquisition of the image data. The fiducial may be incorporated into and/or separate from and a tracking device. Similarly, a fiducial may be integrated the and/or separate from a tracking device associated with any other portion, such as the robotic system 20.

Also or alternatively, a subject tracking device, also referred to as a dynamic reference frame (DRF), may be associated with the subject 30 in block 328. Associating the fiducial in block 328 may include fixing a fiducial to the subject 30. For example, as noted above, the patient tracker 58 may be fixed to the spinous process 130 of the spine 126 of the subject 30. The patient tracker 58 may include fiducial portions and/or tracking portions. The patient tracker 58 may include only one or the other and/or both tracking portions and fiducial portions. Nevertheless, as illustrated in FIGS. 3 and 4, the patient tracker 58 may be positioned relative to the subject 30, such as during image data acquisition. A pose of the patient tracker 58 relative to portions of the subject 30 may, therefore, be determined.

With reference to FIG. 3, the patient tracker 58 may be positioned relative to the subject 30. As illustrated in FIG. 3, various portions of the subject 30 may be modeled and/or imaged. For example, an outer extent geometry or portion 210 may be determined of the subject 30 and/or a model may be generated based upon the data acquired with the imaging system 80. The patient tracker 58 may have a geometry or pose known relative to the outer extent 210. For example, as illustrated in FIG. 3, a distance or geometry cone 340 may be determined between the patient tracking device 58 and of the outer extent 210. With additional reference to FIG. 5, in the subject or navigation space the patient tracker 58 may include a tracking portion point or origin 344. The point 344 may be tracked with an appropriate tracking system, such as those discussed above. Therefore, a pose or distance 340 may be determined between the tracking point 344 and any portion of the external geometry 210 of the subject 30. Thus, the tracked pose of the patient accurate 58 may be determined with of the tracking system relative to an external extent or surface 210 of the subject 30.

The method or process 300 may include various portions, including the inputs or steps as noted above. The process 300 may then include acquiring intraoperative image data in block 360. Acquiring the intraoperative image data in block 360 may include acquisition of image data of any appropriate type of the subject 30. As discussed above, the imaging system 80 may be used to acquire image data of the subject 30. During the acquisition of the image data the pose or position of the imaging system 80 relative to the subject 30 may be determined, such as in use of the O-arm® imaging system, as is understood by one skilled in the art. That is the pose of the portions acquiring the image data of the subject 30 may be known or determined during the acquisition of the image data of the subject 30. In addition and/or alternatively thereto, various tracking portions may be associated with the imaging system 80. By tracking the pose of the imaging system 80 during acquisition of the image data of the subject 30, the image data acquired with the imaging system 80 may be automatically registered relative to the subject 30. In addition, according to various embodiments, the image data may be acquired of the subject 30 at known or determined poses and may be registered automatically to the subject in addition due to the tracking of the patient tracker 58 and tracking the imaging system 80 with a tracker. Accordingly, the image data acquired with the imaging system 80 may be acquired of the subject 30 and include a known or determined pose of the various portions of the image data, such as any surfaces or segmented portions that may be made or determined of the subject 30.

The image data may be registered relative to any preoperative image data, such as that from block 314, in block 364. The intraoperative image data may also be registered to the subject 30 and/or the navigation space in block 364. Registration of the acquired image data from block 360 to the subject 30 may be performed in any appropriate manner. As discussed above, the image data may be acquired at known poses relative to the subject 30. Therefore, the image data may be inherently or automatically registered to the subject 30, as is understood by one skilled in the art. In addition and/or alternatively thereto, the image data acquired in block 360 may be registered to the subject 30 using known registration techniques. For example, portions of the subject 30 may be identified in the image data for registration to any appropriate system, such as the navigation system or navigation space. The image data acquired of the subject 30 may include a fiducial that is imaged with the subject and/or portions of the subject, such as portions of the vertebrae 126.

The image data of the subject may be registered relative to the navigation space and/or any other appropriate portion, such as the robotic system 20. As discussed above, the origin of the robotic system may be determined relative to the subject and/or may be tracked, such as with the robotic system tracker 54. Further, the subject 30 may be tracked with the patient or subject tractor 58. As discussed above, the pose of the subject tractor 58 may be determined relative to one or more portions of the subject 30, such as by determining order recalling a pose of the subject tractor 58 relative to one or more portions of the subject 30, such as the outer extent 210, the vertebrae 126, or other appropriate portions.

In addition to registration of the intraoperative image data to the subject 30, the intraoperative image data may be registered to the pre-operative image data. The registration of the pre- and intra-operative image data may be performed according to known techniques, such as identifying common points. A translation map may then be made between the two. This may allow a pre-operative plan made with the pre-operative image data to be registered or translated to the intra-operative image data that allows the robotic system 20 and other portions to be registered to the subject 30. Thus, the pre-operative image data may also be registered or translated to the subject 30 and navigation space or coordinate system and the robotic space or coordinate system.

The registration of the intraoperative data to the preoperative image data is optional, as illustrated in FIG. 6. Nevertheless, the registration may allow for ensuring that the intraoperative image data is aligned with of the preoperative image data, particularly with a plan thereof. Nevertheless the acquired intraoperative image data may be used for various purposes, as discussed further herein.

The intraoperative image data acquired in block 360 may be used to reconstruct or generate a model of at least a first portion of the subject 30 at a selected resolution, which may be a first resolution in block 370. The intraoperative image data may also be used to generate or reconstruct a second model or image of a second portion of the subject, which may be referred to as a region of interest, at a second resolution and block 374. Initially, the first and second resolution may be the same or different resolutions. The first and second resolutions may be used to generate the two models at selected speeds, clarity for display, refinement or fine positioning as discussed herein, or other appropriate purposes. Accordingly, the reconstruction at a first and second resolution may be at different or the same resolutions based upon various features and considerations, such as a user input. Nevertheless, a reconstruction of at least two portions of the subject 30 may be performed in the respective blocks 370, 374.

The reconstructions in blocks 370, 374 may include any appropriate type of reconstruction. In various embodiments, the reconstruction may be an image, such as of the vertebrae and/or a general volume. For example, the reconstruction may include the image 108. The reconstruction in block 374 may be of a region of interest, such as one or more of the vertebrae. Therefore, the second resolution may be a high resolution or very detailed resolution to allow for viewing of various intricacies of the selected region of interest, such as the vertebrae, as illustrated in FIG. 5. The reconstruction or model generated in block 370 may be of a lower resolution to illustrate a general boundary and/or define a general boundary. For example, the external boundary 210 of the subject 30 may be reconstructed with the image data. The outer boundary 210 may be used for various purposes, such as defining an extent of the subject 30. The external geometry or boundary of the subject 30 may be used when moving the robotic system 20, including the end effector 44 relative to the subject 30 and/or portions connected to the subject 30. For example, the patient tracker 58 may be positioned on the subject 30 and its pose or position may be known relative to the external boundary 210 based upon the tracked pose of the patient tracker 58 and the reconstructed model based upon the image data acquired with the imaging system 80. That is the pose of the subject 30 may be known at the time of the acquisition of the image data and a boundary, such as the external boundary 210, of the subject may be known relative to the patient tracker 58. As a patient tracker 58 may be tracked in the navigation system and the pose of the robot 20, such as the end effector 44, may also be known in the navigation system, the pose of the boundary 210 may be known relative to the end effector 44 to assist in determining possible zones of movement (i.e., Go Zones) of the end effector 44.

Accordingly, a determination of a pose of tracked portions may be made in block 380. The determination of the tracked pose may be based upon tracking the various portions directly, such as tracking the patient tracker 58, the instrument tracker 66, for the robotic tracker 54. Other determinations may be made such as determining the pose of the end effector 44 relative to the origin, such as the base 34, of the robotic system 20. As the robotic system 20 may be registered relative to the navigation coordinate system, as discussed above, knowing the pose of the end effector 44 in the robotic coordinate system may allow for its pose to be determined in the navigation coordinate system. Regardless, determination of pose of the movable portions may be determined in block 380.

A determination of a Go and/or No-Go zones may be made in Block 390. The determination of the Go and/or No-Go zones may include a determination of only the No-Go zone and defining anything not in the No-Go zone as being a Go zone. For the ease of the following discussion, therefore, it is understood that either may occur but the discussion may relate to the determination of the No-Go zones.

The determination of the No-Go zones may be based upon the determination or the reconstruction of the outer boundary 210. Additionally, any tracked portions, such as the patient tracker 58, may also be determined to have a volume that is also within the No-Go zones. Therefore, the No-Go zones, as illustrated in FIG. 5, may be the external boundary of the subject, such as the skin surface of the subject 30. That No-Go zones may also include a volume that extends around the patient tracker, such as the volume 384. Other appropriate portions may also be determined to be in that No-Go zones and may also be temporary or permanent, such as the instrument 68. The instrument 68 may be moved relative to the subject and may be tracked with the navigation system. Therefore, the position of the instrument 68 may be a temporary No-Go zones. In various embodiments, the user may also input selected regions or volumes as No-Go zones. The user input No-Go zones may include selected volumes relative to the subject or other instrumentation in the procedure area that may not be tracked.

Nevertheless, the system, such as the robotic system 20, the navigation system, or other appropriate system may determine the No-Go zones. The determination of the No-Go zones may be a process carried out by a processor or process or system, as is understood by one skilled in the art. The process or system may execute instructions to determine the external geometry based upon that the image data and the reconstructed models based on the image data to determine at least part of no go zoned, such as the outer geometry 210, of the subject 30. The Go and No-Go Zones may be defined as inside, outside, or between any one or more selected boundaries. Thus, the Go and No-Go Zones may be selected three-dimensional volumes between and/or relative to selected boundaries. For example, as a volume within a boundary and/or between two boundaries.

The determination of the No-Go zones, such as based upon the pose of tracked portions and the reconstruction of various models, as discussed above, may be used in determining possible poses and/or possible movements of the robotic system 20, or moveable portions thereof such as the end effector 44 or the arm 40, to ensure that they do not pass through or stop in the No-Go zones. Accordingly, the process 300, may include a determination of the robotic Go zone in block 394. As discussed above, the robotic Go Zone may include any portion that does not include a No-Go zones. Therefore determination of the robotic Go Zone in block 394 may be a determination of any possible range of motion of the robotic system that does includes only a Go zones determine a block 390 and does not include a No-Go zones.

The process 300, therefore, may also be an input and/or include a subroutine for moving the robot and subroutine 404. The subroutine 404 may be a portion of the process 300 and and/or be separate therefrom. Therefore, the subroutine 400 may be executed solely by the robotic system based upon inputs from other systems, such as the navigation system. Regardless, the subroutine 404 may allow for movement of the robotic system only within the Go zone and not within the No-Go zones.

For example, a query or check of whether the robot is to be moved may be made in Block 410. If the robot is not moved a NO path 414 may be followed to repeat the determination of whether the robot is to be moved by 410 and/or determine the go zoning block 394.

If the robot is determined to be moved, however, a YES path 420 may be followed to determine an initial pose of the robotic black 424. The initial pose may be recalling of a last pose, a tracking of the current pose, such as with the navigation system, a tracking of the current pose such as with the robotic system 20, or other appropriate determination. In various embodiments, for example, the initial or current pose may be input by the user. It is understood, however, that the determination or evaluation of the initial current pose may be automatic, manual, or combination thereof.

A determination of a final pose may be made in block 430. The final pose may be a final pose or position for the end effector 44. For example, during a first portion of a procedure, the end effector 44 may be used to guide an instrument to position a first pedicle screw. After a period of time, such as after positioning the first pedicle screw with the end effector 44 in the first pose, it may be selected to insert a second pedicle screw with the end effector 44 at a second pose. Therefore, the determination of the second pose of the robotic system 20 may include also determining a path between the initial or first pose and the second pose in block 434. In determining a path, a straight line may be evaluated between the first pose in the second pose. The straight-line may be evaluated as to whether it is only in the Go zone in block 434. If it is determined that to the straight-line path is not only in the Go zone or includes portions in the No-Go zones, a second path may be determined. The process may, therefore, be iterative to determine a path, which may be an efficient or optimal path, from the initial or first pose to the second pose in block 434. Regardless, the path may be determined only in the Go zone in block 434. By determining the path to be only on the Go zone, the robotic system 20, including the end effector 44 or portions of the robotic arm 40, may be determined to not contact or not interfere with portions of the subject 30, the patient tractor 58, or other portion selected to be in the no go zone as discussed above. While it may be selected to move the arm 40 or portions thereof only in the Go Zone, it is understood that selected movements in the No-Go zones may be selected automatically and/or with user input or override.

The path may be output and block 440. The output path may be output based upon the determination of the path only in the Go Zone in block 434. The path may be output and stored and/or used to control movement of the robotic system in block 444. Therefore, the path output in block 440 may be transmitted from a selected processor system and/or determined with the processor system of the robotic system 20 to control movement of the robotic arm 40. The control the movement may include speed, amount, and position, of movement or driving of the robotic drive mechanisms in the robotic arm 40 or other appropriate outputs. In various embodiments, for example, the output for the path may include a manual movement of the robotic arm 40 if the robotic arm 40 is not able to pass through a Go zone only path. Therefore, it is understood that to the output path may include both automatic movements of the robotic arm 40 and/or outputs for manual movements of the robotic arm 40.

Regardless, the process 300 and/or the sub-process 404 may be used to determine a possible movement or path of the robotic arm 40, including the end effector 44, that may move the end effector 44 without passing through a No-Go zone. This may allow the robotic arm, including the end effector 44, to be moved relative to the subject 30 without contacting the subject 30 and/or interfering with of the procedure in a selected manner.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications or connections may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method for determining movement of and moving at least a moveable portion of a robotic system to at least minimize contact with portions exterior to the robotic system, comprising:
acquiring image data of a subject;
generating a model of the subject based at least on the acquired image data;
tracking a reference marker, connected to the subject, with a tracking system;
determining a volume defined by the subject based on the generated model;
defining a no-go region relative to the determined volume; and
tracking the moveable portion relative to the defined no-go region.

2. The method of claim 1, further comprising:
tracking the reference marker to determine a pose of at least one of the determined volume defined by the subject or the defined a no-go region.

3. The method of claim 2, further comprising:
registering a robotic coordinate system to the subject, wherein the subject at least in part defines the no-go region.

4. The method of claim 1, further comprising:
registering a robotic coordinate system to an image space defined by the generated model;
registering the image space to a navigation space;
wherein tracking the moveable portion relative to the defined no-go region includes tracking the moveable portion in the navigation space.

5. The method of claim 4, further comprising:
displaying a graphical representation of the moveable portion relative to an image.

6. The method of claim 1, further comprising:
performing at least a first portion of a procedure with the moveable portion in a first position at a first time;
moving the moveable portion to a second position at a second time; and
determining a path of movement of the moveable portion to the second position from the first position that does not pass through the no-go region.

7. The method of claim 6, wherein determining the path of movement of the moveable portion to the second position from the first position that does not pass through the no-go region comprises:
determining a first path;
evaluating whether the determined first path passes through the no-go region; and
determining a second path when the first path is evaluated to pass through the no-go region.

8. The method of claim 6, further comprising:
outputting the determined path to control movement of the moveable portion including providing commands to drive mechanisms.

9. The method of claim 8, further comprising:
receiving an input from the user regarding the no-go region.

10. The method of claim 6, further comprising:
operating the robotic system to move the moveable portion to move alone the determined path.

11. The method of claim 10, further comprising:
positioning an end effector of the moveable portion via the determined path.

12. The method of claim 1, further comprising:
providing the robotic system relative to the subject.

13. A system for determining movement of and moving at least a moveable portion of a robotic system to at least minimize contact with portions exterior to the robotic system, comprising:
a tracking system;
a reference marker, connected to a subject, configured to be tracked with the tracking system;
a processor configured to execute instructions to:
acquire image data of the subject;
generate a model of the subject based at least on the acquired image data;
determine a volume defined by the subject based on the generated model;
define a no-go region relative to the determined volume; and
determine a pose of the moveable portion relative to the defined no-go region.

14. The system of claim 13, further comprising:
the robotic system configured to be fixed relative to the subject.

15. The system of claim 14, further comprising:
a robotic system tracked configured to be tracked by the tracking system to allow the determine the pose of the moveable portion.

16. The system of claim 13, further comprising:
an imaging system configured to capture the image data.

17. The system of claim 13, wherein the processor system to define the no-go region relative to the determined volume includes determining an external geometry of the subject based on the acquired image data.

18. A method for determining movement of and moving at least a moveable portion of a robotic system to at least minimize contact with portions exterior to the robotic system, comprising:
positioning the robotic system relative to the subject.
acquiring image data of a subject with an imaging system;
executing instructions with a processor to:
reconstruct a model of the subject based at least on the acquired image data;
determine an external geometry of the subject based on the reconstructed model;
define a go region relative to the determined external geometry;
receiving at least an end point pose of the moveable portion;
determine a path from a current pose of the moveable portion to the end point pose that moves the moveable portion only in the go region; and
outputting the determined path.

19. The method of claim 18, wherein determine a path from a current pose of the moveable portion to the end point pose that moves the moveable portion only in the go region comprises:
executing further instructions with the processor to:
determine a first path;
evaluate whether the determined first path passes through only the go region; and
determine a second path when the first path is evaluated to pass through a region other than the go region.

20. The method of claim 18, further comprising:
receiving an input from the user regarding the go region.
